(19) United States
(12) Patent Application Publication
(10) Pub. No.: US 2008/0200918 A1
(43) Pub. Date: Aug. 21, 2008

(54) PEDICLE SCREW DRIVER

(76) Inventors: James Spitler, Plano, TX (US); Michael Castro, Uxbridge, MA (US); Rex Shores, Norfolk, MA (US)

Correspondence Address: IBM CORPORATION (CS);C/O CARR LLP, 670 FOUNDERS SQUARE, 900 JACKSON STREET, DALLAS, TX, 75202, US

(21) Appl. No.: 12/029,374

(22) Filed: Feb. 11, 2008

Related U.S. Application Data
(60) Provisional application No. 60/889,402, filed on Feb. 12, 2007.
(60) Provisional application No. 60/889,797, filed on Feb. 14, 2007.

(51) Int. Cl. A61B 17/58; B21J 15/10; A61B 17/56

(52) U.S. Cl. 606/104; 227/53; 606/301

(57) ABSTRACT

A surgical driver includes an elongated drive shaft engageable with a surgical bone anchor in torque transmitting relationship, a locking sleeve, and a sleeve locking member. The locking sleeve is mounted to the drive shaft and is engageable with the head of surgical bone anchor. The sleeve locking member is mounted for relative motion between a first position in which it is simultaneously engaged with both the drive shaft and the locking sleeve in rotationally fixed relationship and a second position in which it is disengaged from the rotationally fixed relationship with at least one of the drive shaft and locking sleeve.

### CROSS-REFERENCED APPLICATIONS

This application relates to, and claims the benefit of the filing date of, co-pending U.S. provisional patent application Ser. No. 60/889,402 entitled Pedicle Screw Driver, filed Feb. 12, 2007, the entire contents of which are incorporated herein by reference for all purposes. This application also relates to, and claims the benefit of the filing date of, co-pending U.S. provisional patent application Ser. No. 60/889,797 entitled Pedicle Screw Driver, filed Feb. 14, 2007, the entire contents of which are incorporated herein by reference for all purposes.

### TECHNICAL FIELD

The invention relates to tools for bone stabilization systems, and more particularly to tools for inserting screws into bones.

### BACKGROUND OF THE INVENTION

The human spine provides a vast array of functions, many of which are mechanical in nature. The spine is constructed to allow nerves from the brain to pass to various portions of the middle and lower body. These nerves, typically called the spinal cord, are located in a region within the spine called the spinal canal. Various nerve bundles emerge from the spine at different locations along the lateral length of the spine. In a healthy spine, these nerves are protected from damage and/or undue pressure thereon by the structure of the spine itself.

The spine has a complex curvature made up of a plurality of individual vertebrae separated by intervertebral discs. These discs hold the vertebrae together in a flexible manner so as to allow a relative movement between the vertebrae from front to back and from side to side. This movement then allows the body to bend forward and backward, to bend from side to side, and to rotate about a vertical axis. Throughout this movement, when the spine is operating properly the nerves are maintained clear of the hard structure of the spine.

Over time, or because of accidents, the intervertebral discs loose height, become cracked, dehydrated, or herniated. The result is that the disc height is reduced leading to compression of the nerve bundles, causing pain and in some cases damage to the nerves.

Currently, there are many systems and methods at the disposal of a physician for reducing, or eliminating, the pain by minimizing the stress on the nerve bundles. In some instances, the existing disk is removed and an artificial disk is substituted therefore. In other instances, two or more vertebrae are fused together to prevent relative movement between the fused discs.

Often there is required a system and method for maintaining, or recreating, proper space for the nerve bundles that emerge from the spine at a certain location. In some cases a cage or bone graft is placed in the disc space to preserve, or restore, height and to cause fusion of the vertebral level. As an aid in stabilizing the vertebrae, one or more rods or braces are placed between the fused vertebrae with the purpose of the rods being to support the vertebrae, usually along the posterior of the spine, while fusion takes place. These rods are often held in place by anchors which are fitted into the pedicle of the vertebrae. One type of anchor is a pedicle screw, and such screws come in a variety of lengths, diameters, and thread types.

A polyaxial pedicle screw may include an anchor shaft and a separate polyaxial head movably attached to the anchor shaft. The polyaxial head attaches to the rods and the relative movement between the polyaxial head and anchor shaft facilitates alignment of the rods with the pedicle screws. Various instruments have been developed in an attempt to insert the rod, polyaxial head, and anchor shaft in a reliable and efficient manner. Once the rods and screws are positioned, a connecting mechanism, such as a locking cap may connect the rod, polyaxial head, and anchor shaft.

### SUMMARY

The present invention provides a surgical screw driver for bone anchors.

In one aspect of the invention, the surgical screw driver may include an elongated drive shaft engageable with the pedicle screw in torque transmitting relationship, a locking sleeve, and a sleeve locking member. The locking sleeve is mounted to the drive shaft and is engageable with the head of the pedicle screw. The sleeve locking member is mounted for relative motion between a first position in which it is simultaneously engaged with both the drive shaft and the locking sleeve in rotationally fixed relationship and a second position in which it is disengaged from the rotationally fixed relationship with at least one of the drive shaft and locking sleeve.

In another aspect of the invention, the surgical screw driver is operable to pretension a pedicle screw into axial engagement with the pedicle screw driver by drawing the screw head axially toward the drive shaft.

In another aspect of the invention, the pedicle screw driver includes an axially captured protective sleeve coaxially mounted to the pedicle screw driver for relative rotation. The protective sleeve is held stationary while the rest of the instrument rotates. The protective sleeve prevents abrasion, snagging, and wrapping up of objects adjacent the surgical site including the margins of the incision, surgical drapes, and the user's glove.

### BRIEF DESCRIPTION OF THE DRAWINGS

Various examples of the present invention will be discussed with reference to the appended drawings. These drawings depict only illustrative examples of the invention and are not to be considered limiting of its scope.

FIG. 1 is a perspective view of a pair of pedicle screws inserted into adjacent vertebrae and joined by a rod;

FIG. 2 is a perspective view of one of the pedicle screws of FIG. 1;

FIG. 3 is a top plan view of the pedicle screw of FIG. 2;

FIG. 4 is a side sectional view of the pedicle screw of FIG. 2 taken along line 4-4 of FIG. 3;

FIG. 5 is a perspective view of a pedicle screw driver assembled with the screw of FIG. 2;

FIG. 6 is a side sectional view of the pedicle screw driver of FIG. 5 taken along line 6-6;

FIG. 7 is an exploded perspective view of the pedicle screw driver of FIG. 5;

FIG. 8 is a perspective view of the pedicle screw driver of FIG. 5;

FIG. 9 is a perspective view of a component of the pedicle screw driver of FIG. 5;

FIG. 10 is a perspective view of a component of the pedicle screw driver of FIG. 5; and

FIG. 11 is a perspective view of a component of the pedicle screw driver of FIG. 5.

### DESCRIPTION OF THE ILLUSTRATIVE EXAMPLES

In the following discussion, numerous specific details are set forth to provide a thorough understanding of the present invention. However, those skilled in the art will appreciate that the present invention may be practiced without such specific details. In other instances, well-known elements have been illustrated in schematic or block diagram form in order not to obscure the present invention in unnecessary detail. Additionally, for the most part, minor details have been omitted inasmuch as such details are not considered necessary to obtain a complete understanding of the present invention, and are considered to be within the understanding of persons of ordinary skill in the relevant art.

Embodiments of a screw driver may include a drive member engageable with the pedicle screw in torque transmitting relationship. For example, the drive member may include a drive shaft having a torque input end and a torque output end. The torque input end may have a handle able to be directly gripped by a user and rotated to transmit torque to the torque output end. The torque input end may have a drive adapter for coupling the drive member to another instrument in torque transmitting relationship. Such other instruments may include a handle, a ratcheting handle, a manually operated brace, a gear reduction mechanism, a powered rotary instrument, and/or other suitable instruments. For example, the torque input end may include a drive adapter for engaging a powered rotary driver in positive torque transmitting relationship.

The torque output end may include a male portion that engages a female portion of the pedicle screw, a female portion that engages a male portion of the pedicle screw, and/or any other suitable torque transmitting engagement mechanism. The engagement may include a positive torque transmitting geometry such as a rotationally keyed interfering geometry. For example, the engagement may include male/female geometric pairs. Examples include blade and slot engagements, triangular engagements, square engagements, pentagonal engagements, hexagonal engagements, and/or any suitable polygonal engagement with any number of sides. The engagement may include a regular polygonal shape, star shapes, splines, and/or any other suitable torque transmitting shape. For example, the pedicle screw may include a male star-shaped portion and the driver may include a corresponding female star-shaped portion.

The screw driver may include a second member engageable with a polyaxial head to align the polyaxial head generally axially parallel to the anchor shaft during insertion of the pedicle screw. The second member may engage the polyaxial head in threaded, snap-fit, press-fit, taper fit, and/or other suitable engagement. The second member may engage the polyaxial head in axial force transmitting engagement. The second member may be mounted coaxially, non-coaxially, parallelly, transversely, and/or otherwise mounted relative to the drive member. The second member may include a solid rod, a hollow tube, and/or other suitable structures. For example, the second member may include a sleeve mounted coaxially over the drive shaft and having a threaded portion engageable with the polyaxial head in axial force transmitting relationship to align the polyaxial head coaxially with the anchor shaft and draw the pedicle screw and drive shaft together to lock the drive shaft to the anchor shaft in torque transmitting relationship.

The second member may include a locking mechanism to prevent disengagement of the second member from the polyaxial head. The locking mechanism may lock the second member rotationally and/or axially relative to the drive member. The locking mechanism may include a sleeve locking member that simultaneously engages both the drive member and the second member to prevent relative movement between them. For example, the sleeve locking member may be releasably engageable with both the drive member and second member in torque transmitting relationship to prevent relative rotation of the drive member and the second member. The engagement between the sleeve locking member and the other members may include a frictional engagement and/or a positive engagement. A positive engagement may include polygonal, splined, and/or any suitable positive torque transmitting engagement. The sleeve locking member may be movable between a position in which it is engaged with both the drive member and the second member and a position in which it is disengaged from one or both of the drive member and second member. For example, the sleeve locking member may be mounted for axial translation between a first position in which it positively engages both the drive member and second member in torque transmitting relationship and a second position in which it is disengaged from at least one of the drive member and second member.

The screw driver may include an outer portion that remains stationary relative to the surgical site to isolate the rotating drive member from the surgical site. For example, the drive shaft may be coaxially mounted for relative rotation inside an outer sleeve. The outer sleeve may form a surface grippable by a user.

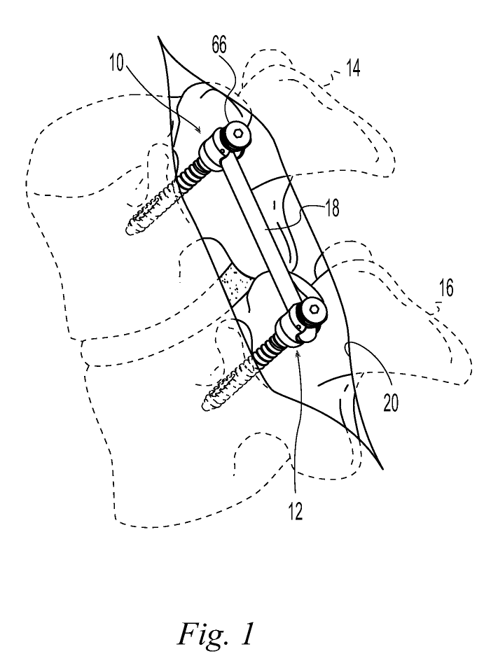

FIG. 1 illustrates a pair of pedicle screws 10, 12 inserted in adjacent vertebrae 14, 16 and connected by a fixation rod 18. The screws 10, 12 and rod 18 are placed through an incision 20. In a minimally invasive surgical procedure, this incision 20 can be quite small. Furthermore, a pair of incisions, each just large enough to receive a pedicle screw, can be substituted for the single incision. The margins of the incision tend to press in around the surgical site which may interfere with the insertion of the screws 10 and 12.

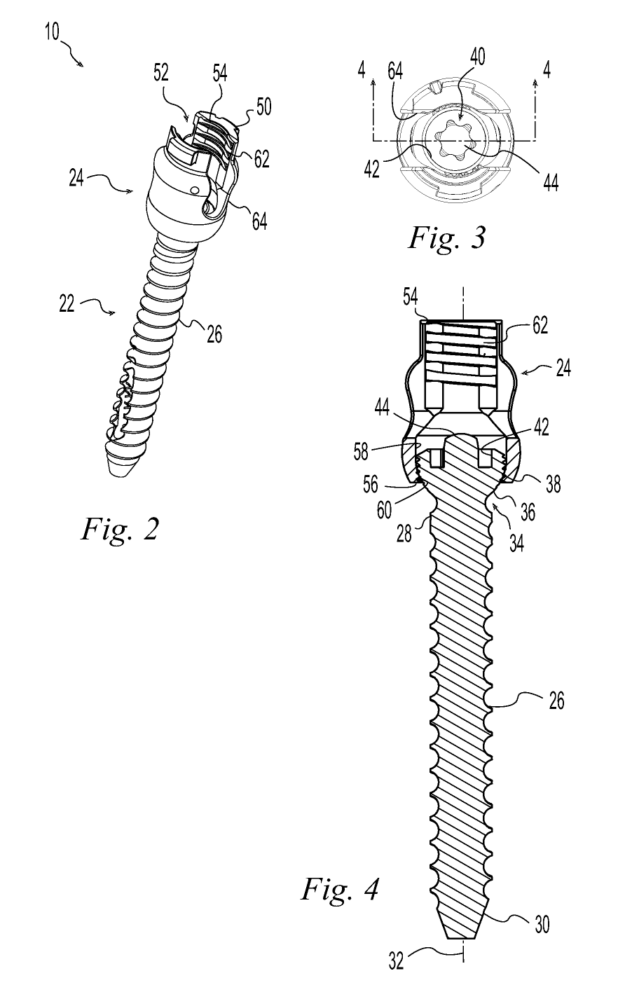

FIGS. 2-4 depict one of the pedicle screws 10, 12 of FIG. 1 in detail. The pedicle screw 10 may include an anchor member 22 and a separate polyaxial head member 24. The anchor member 22 may have an elongated threaded shaft 26 having a proximal end portion 28, a distal end portion 30 and a longitudinal axis 32 extending therebetween. The anchor member 22 may define a head 34 at the proximal end portion 28 that may have a spherical outer surface 36 with an external, male, screw thread 38. The head may define a drive mechanism 40 (FIG. 3) including an axial bore 42 and a coaxial drive post 44. The drive post 44 may define a star-shaped male engagement portion engageable with a driver in torque transmitting relationship.

The polyaxial head member 24 may include a generally cylindrical hollow body 50 having an axial through bore 52 extending from a proximal opening 54 to a distal opening 56. The through bore 52 may also define an internal spherical seating surface 58 adjacent to the distal opening 56. The through bore 52 may further define an internal, female, screw thread 60 at the distal opening 56. The through bore 52 may define an internal, female, screw thread 62 adjacent to the proximal opening 54. A transverse channel 64 may extend through the polyaxial head member 24 intermediate the proximal and distal openings 54, 56. The transverse channel 64 may receive the rod 18. The screw thread 62 adjacent to the proximal opening 54 may receive a set screw 66 (FIG. 1) which may secure the rod 18, polyaxial head member 24, and anchor member 22 together.

The anchor member 22 and the polyaxial head member 24 may be assembled by engaging the female screw thread 60 at the distal opening 56 of the polyaxial head member 24 with the male screw thread 38 of the anchor member head 34. The polyaxial head member 24 may be threadably advanced until the female thread 60 passes completely over the male thread 38 to a fully assembled position. In the fully assembled position, the spherical outer surface 36 of the anchor member head 34 is articulable on the spherical seat 58 of the polyaxial head member 24 to permit varying the angle between the anchor member 22 and polyaxial head member 24. The angular movement of the polyaxial head member 24 relative to the anchor member 22 may increase the difficulty of maintaining axial alignment and control during insertion of the anchor member 22.

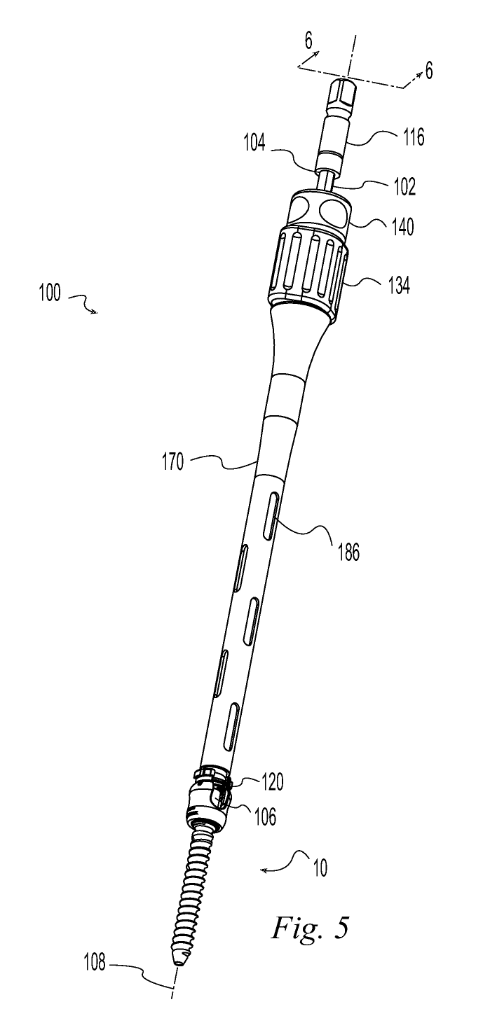

FIGS. 5-11 depict an exemplary screw driver 100 which may be used with the pedicle screws 10, 12 of FIGS. 1-4, or any other type of screw or bone anchor. The screw driver 100 may not be limited to just the insertion of pedicle screws, but may be utilized to implant screws in other areas of the spine and the body. The driver 100 may include an elongated drive shaft 102 having a proximal torque input end portion 104, a distal torque output end portion 106, and a longitudinal axis 108 extending therebetween. The distal end portion 106 may define a drive mechanism 110 including an axial bore 112. The bore 112 may define a complimentary star-shaped female engagement portion 114 engageable with the drive post 44 of the anchor member 22 in torque transmitting relationship. A drive adapter 116 may be connected to the proximal end 104 of the drive shaft 102 and may provide a mechanism for coupling the drive shaft 102 to another instrument in torque transmitting relationship. In the exemplary embodiment, the proximal end portion 104 of the drive shaft 102 may have a “D”-shaped cross section and the drive adapter 116 and drive shaft 102 may be joined together to form a single member.

The screw driver 100 may include a tubular member, or locking sleeve 120, coaxially mounted over the drive shaft 102 for rotation relative to the drive shaft 102. The locking sleeve 120 may include a proximal end portion 122, a distal end portion 124, and an axis 126 extending therebetween. The locking sleeve 120 may include a through bore 121 defining an internal shoulder 128 that abuts an external shoulder 130 on the drive shaft 102 which may limit axial translation of the locking sleeve 120 relative to the drive shaft 102. The abutting shoulders 128, 130 may cause the distal end portion 106 of the drive shaft 102 to extend at least a predetermined distance 132 from the distal end portion 124 of the locking sleeve 120. The locking sleeve 120 may define a locking sleeve grip member 134 adjacent the proximal end portion 122 of the locking sleeve 120 to facilitate rotating the locking sleeve 120. In the illustrative embodiment, the grip member 134 may include a flattened cylinder coaxially mounted to the locking sleeve 120. The grip member 134 may be permanently or rigidly joined to the locking sleeve 120 to form a single member. The through bore 121 of the locking sleeve may extend completely through the grip member 134. An axial counter bore 136 (FIG. 11) may extend distally into the grip member 134 and define a series of lock members or longitudinal grooves 138 in the bore wall. The distal end portion 124 of the locking sleeve 120 may define a coupling mechanism 137 engageable with the screw thread 62 adjacent to the proximal opening 54 of the polyaxial head member 24. The coupling mechanism 137 may include an external or internal geometry, such as a male screw thread (as shown) or may include other geometries capable of coupling to a screw head. The locking sleeve 120 may also include optional elongated openings 139 through the sidewall of the locking sleeve 120 which may facilitate cleaning debris from the screw driver 100.

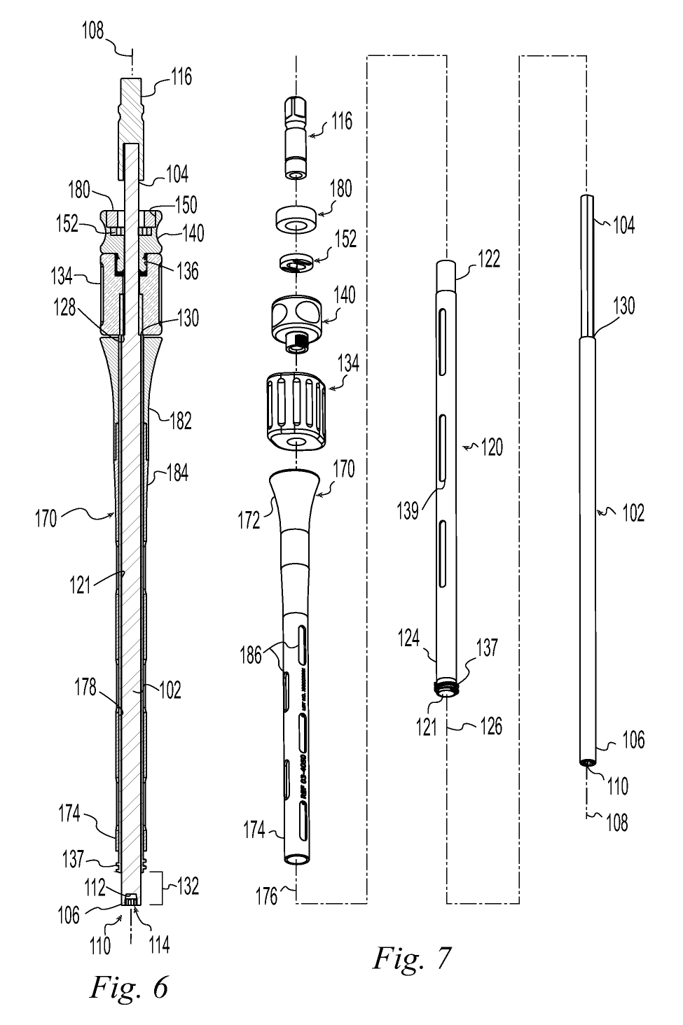

The screw driver 100 may include a locking mechanism for releasably locking the rotational position of the locking sleeve 120 relative to the drive shaft 102. The locking mechanism may include a sleeve lock 140 (FIG. 9) having a generally cylindrical body 142, a distal knob or projection 143, and an axial through bore 144. The through bore 144 may include a flat portion 146 causing the through bore 144 to be “D”-shaped. The “D”-shaped through bore 144 may engage the corresponding “D”-shaped cross section on the proximal end 104 of the drive shaft 102 to prevent relative rotation between the sleeve lock 140 and the drive shaft 102 while permitting axial relative translation between the sleeve lock 140 and the drive shaft 102. The knob 143 may define counter lock members or projections such as external splines 148 axially engageable with the grooves 138 of the grip member 134 to prevent relative rotation between the sleeve lock 140 and the grip member 134 of the locking sleeve 120. The sleeve lock 140 may further define an axial counter bore 150 (FIG. 6) extending distally into the body 142. A friction plate or disk member 152 (FIG. 10) may be inserted into the through bore to provide a frictional grip between the sleeve lock 140 and the drive shaft 102 to resist relative sliding between the sleeve lock 140 and drive shaft 102. The friction plate 152 may include a generally cylindrical body 154 having a central axial through opening 156 sized to press fit onto the drive shaft 102. The body 154 may further include radially offset axial through openings 158, 160 defining flexible webs 162, 164. When the friction plate 152 is pressed over the drive shaft 102, the flexible webs 162, 164 expand outwardly into the offset openings 158, 160 to provide a resilient grip on the drive shaft 102. The flexible webs 162, 164 may be optional on the friction plate 152, for example the friction plate 152 may be composed of a generally soft flexible material which may be sized to grip the drive shaft 102. The friction plate 152 may be secured in the counter bore 150 by a retainer 180 (FIG. 7) in the form of a hollow cylinder that is press fit into the counter bore 150. The friction plate may be any size or shape and may be made of any suitable material. In the illustrative pedicle screw driver, the friction plate is made of a polymer.

The screw driver 100 may include an elongated hollow protective sleeve 170 coaxially mounted or positioned over the locking sleeve 120 for rotation relative to the locking sleeve 120. The protective sleeve 170 may include a proximal end 172, a distal end 174, and a longitudinal axis 176 extending therebetween. The protective sleeve may define a longitudinal through bore 178 having a diameter smaller than the grip member 134 of the locking sleeve 120 and smaller than the polyaxial head engaging threads 137 of the locking sleeve 120. The protective sleeve 170 may mounted on the locking sleeve 120 with the proximal end 172 of the protective sleeve 170 distal to the grip member 134 and the distal end 174 of the protective sleeve 170 proximal to the thread 137 such that the protective sleeve is captured on the locking sleeve 120. The outer surface 181 of the protective sleeve 170 may flare outwardly proximally to match the minor diameter of the grip member 134 and smooth the transition between the protective sleeve 170 and the grip member 134. The illustrative protective sleeve 170 may be assembled from a proximal piece 182 and a distal piece 184 permanently joined into a unitary sleeve to reduce waste during manufacture. The protective sleeve 170 may also include optional elongated openings 186 through the sidewall of the sleeve 170 to facilitate cleaning debris from the screw driver 100 and to improve a user's grip on the protective sleeve.

In the fully assembled screw driver 100 (FIGS. 5, 6, 8), all of the separate components described above and shown exploded in FIG. 7 are captured together in an integrated instrument. The various components of the screw driver 100 may be made from a variety of materials including metals, polymers, ceramics, and/or other suitable materials and combinations thereof.

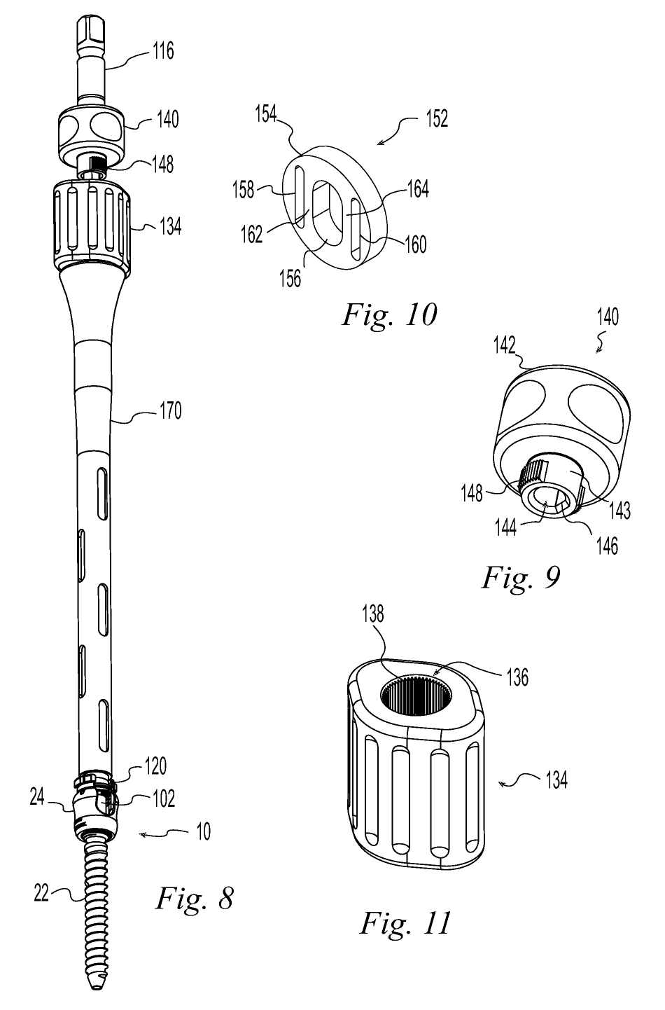

In use, the distal end 106 of the drive shaft 102 may be pressed axially into engagement with the drive mechanism 40 of the pedicle screw 10. The sleeve lock 140 may have a first position and a second position. For example, the sleeve lock 140 may be retracted proximally to a first or unlocked position of FIG. 8 to disengage the sleeve lock splines 148 from the locking sleeve grip grooves 138. The grip member 134 may be rotated to thread the locking sleeve 120 distal screw thread 137 into the proximal screw thread 62 of the polyaxial head member 24. Since the shoulders 128, 130 of the drive shaft 102 and locking sleeve 120 maintain at least a minimum axial extension 132 of the drive shaft 102 from the locking sleeve 120, further engagement of the locking sleeve 120 with the polyaxial head member 24 draws the pedicle screw 10 proximally relative to the drive shaft 102. Thus, rotation of the locking sleeve 120 may cause the drive shaft 102 and pedicle screw 10 to be pressed axially into engagement with one another. Further tightening of the locking sleeve 120 pretensions the driver and screw assembly. The axial tension created by the locking sleeve maintains axial alignment between the polyaxial head member 24 and the anchor member 22 and locks the screw driver 100 to the pedicle screw 10 in torque transmitting relationship. When the desired tension is achieved, the sleeve lock 140 may be slid distally into the locked or second position of FIG. 5 to engage the splines 148 of the sleeve lock 140 with the grooves 138 of the locking sleeve grip member 134. In this position the sleeve lock 140 may be rotationally keyed to both the locking sleeve 120 and the drive shaft 102 and may prevent relative rotation between them.

The pedicle screw driver 100 may be coupled to a drive handle or other instrument via the drive adapter 116. The screw driver 100 may be at least partially inserted through the incision 20 and rotated to drive the pedicle screw 10 into the vertebra 14. During insertion, the user may grip the protective sleeve 170. The protective sleeve 170 remains stationary while the drive shaft 102 and pedicle screw 10 rotate thereby preventing abrasion, snagging, and/or wrapping up of objects adjacent the surgical site including the margins of the incision, surgical drapes, and the user's glove. The protective sleeve 170 may be leveraged against the incision margins to displace the incision while preventing the drive shaft 102 from abrading the incision margins. The tension created by the locking sleeve 120 keeps the drive shaft 102 engaged with the pedicle screw 10 even while the protective sleeve 170 is leveraged against the incision.

When the pedicle screw 10 is fully inserted, the sleeve lock 140 may be slid proximally into the unlocked position, the locking sleeve 120 is unthreaded from the polyaxial head member 24, and the screw driver 100 is removed.

Although examples of a screw driver and its use have been described and illustrated in detail, it is to be understood that the same is intended by way of illustration and example only and is not to be taken by way of limitation. Accordingly, variations in and modifications to the screw driver and its use will be apparent to those of ordinary skill in the art, and the following claims are intended to cover all such modifications and equivalents.

What is claimed is:

1. A surgical instrument for inserting a bone anchor having an anchor member and a polyaxial head member mounted to the anchor member, the anchor member having a proximal end defining a drive mechanism and a distal end, the polyaxial head member being mounted adjacent the proximal end, the surgical instrument comprising:
an elongated drive shaft having a proximal end, a distal end, and a longitudinal axis extending therebetween, the distal end defining a drive mechanism engageable with the drive mechanism of the pedicle screw in torque transmitting relationship;
a locking sleeve mounted to the drive shaft, the sleeve having a proximal end, a distal end, and a longitudinal axis extending therebetween, the distal end defining an engagement portion engageable with the polyaxial head in rotating locking relationship; and
a sleeve locking member mounted for relative motion between a first position in which it is simultaneously engaged with both the drive shaft and the locking sleeve in rotationally fixed relationship, and a second position in which it is disengaged from the rotationally fixed relationship with at least one of the drive shaft and locking sleeve.

2. The surgical instrument of claim 1 wherein the locking sleeve includes a screw thread formed adjacent to its distal end, the screw thread being threadably engageable with the polyaxial head member in axial force transmitting relationship.

3. The surgical instrument of claim 2 wherein the locking sleeve is coaxially mounted to the drive shaft, the locking sleeve having a proximal portion having an internal diameter and a distal portion having an internal diameter, the internal diameter of the proximal portion being smaller than the internal diameter of the distal portion, the proximal and distal portions of the locking sleeve defining a distally facing internal shoulder spaced a predetermined distance from the distal end of the locking sleeve, the drive shaft having a proximal portion having an external diameter and a distal portion having an external diameter, the external diameter of the drive shaft proximal portion being smaller than the external diameter of the drive shaft distal portion, the proximal and distal portions of the drive shaft defining a proximally facing shoulder spaced a predetermined distance from the proximal end of the drive shaft, the shoulder spacing of the drive shaft being greater than the shoulder spacing of the locking sleeve, the proximally facing shoulder abutting the distally facing shoulder such that the drive shaft protrudes from the locking sleeve a predetermined minimum distance.

4. The surgical instrument of claim 3 wherein the sleeve locking member is mounted to the drive shaft for axial translation along a portion of the drive shaft and is simultaneously keyed to the drive shaft in rotationally fixed relationship and further wherein the sleeve locking member is translatable between a first position in which it is keyed to the locking sleeve in rotationally fixed relationship and a second position in which it is disengaged from the locking sleeve.

5. The surgical instrument of claim 4 wherein the sleeve locking member includes an axially extending annular nipple defining an external surface and the locking sleeve defines an axially extending annular counter bore sized to receive the nipple, the nipple and counter bore defining a positively interlocking anti-rotational engagement between them, the nipple and counter bore being engaged when the sleeve locking member is in the first position and disengaged when the sleeve locking member is in the second position.

6. The surgical instrument of claim 4 wherein the sleeve locking member includes an axial through bore engaged with the drive shaft for axial translation and at least one axially offset through bore defining a resilient web between the axial through bore and the offset through bore, the resilient web being biased against the drive shaft.

7. The surgical instrument of claim 6 further comprising a polymer disk having an axial through bore and offset through bore defining the resilient web.

8. The surgical instrument of claim 4 wherein the sleeve locking member includes an non-circular opening axially through the sleeve locking member and the drive shaft includes a portion with a corresponding non-circular cross sectional shape received for relative translation in the non-circular opening and further wherein the sleeve locking member and locking sleeve define an axially engageable and disengageable spline and groove relationship.

9. The surgical instrument of claim 1 further comprising:
a protective sleeve coaxially mounted to the locking sleeve for relative rotation, the protective sleeve having a through bore defining a protective sleeve internal diameter, the locking sleeve having a proximal portion having an external diameter larger than the protective sleeve internal diameter and the locking sleeve having a distal portion having an external diameter larger than the protective sleeve internal diameter, the protective sleeve being axially captured on the locking sleeve.

10. A surgical instrument for inserting a bone anchor having an anchor member and a head mounted to the anchor member, the anchor member having a proximal end and a distal end, the head being mounted adjacent the proximal end, the surgical instrument comprising:
means for transmitting torque to the pedicle screw anchor member;
means for rotatably engaging the head and transmitting an axial force to the head, the means for rotatably engaging being operative to draw the head and anchor member axially toward the means for transmitting torque and pretension the anchor member against the means for transmitting torque; and
means for locking the means for transmitting torque and the means for rotatably engaging in fixed relative rotational relationship, the means for locking being movable between a locked position and an unlocked position.

11. A surgical instrument for inserting a bone anchor comprising:
a tubular member including a proximal end portion and a distal end portion having a coupling mechanism;
a drive shaft positioned within the tubular member and including a proximal end portion having at least one flat side portion and a distal torque output end portion extending distally of tubular member;
a grip member joined to the proximal end portion of the tubular member wherein the grip member includes an inner wall portion defining a bore extending distally into the grip member and one or more lock members extending from the inner wall portion;
a sleeve lock adjacent the grip member including:
a body having an inner wall that defines a axial counter bore that extends distally into the body;
a distal knob portion coupled to the body and positioned at least partially within the bore of the grip member, the distal knob including one or more counter lock members engaged with the lock members;
an inner wall defining an axial through bore that extends into the body and the distal knob wherein at least a portion of the inner wall has a flat portion.

12. The surgical instrument of claim 11 further comprising a tubular sleeve member adjacent the grip member and rotationally positioned at least partially around the tubular driver wherein the coupling mechanism of the tubular driver extends distally of the tubular sleeve member.

13. The bone anchor insertion instrument of claim 12 further comprising a friction member positioned within the axial counter bore of the body and having inner wall defining a central opening which is frictionally engaged with the second driver.

14. The bone anchor insertion instrument of claim 13 further comprising an adapter spaced apart from the sleeve lock and having a first end portion detachably coupled to a handle and a second end portion rigidly joined to a proximal end portion of the second driver.

15. The bone anchor insertion instrument of claim 11 wherein the lock members include axial grooves.

16. The bone anchor insertion instrument of claim 11 wherein the counter lock members include axial splines.

17. The bone anchor insertion instrument of claim 11 wherein the second driver is free to axially translate relative to the tubular driver.

18. A combination of a surgical screw and a surgical driver, the combination comprising:
a surgical screw having an anchor member and a polyaxial head member mounted to the anchor member, the anchor member having a proximal end defining a drive mechanism and a distal end, the polyaxial head member being mounted adjacent to the proximal end for multi-axial rotation; and
a surgical screw driver having a drive shaft, a locking sleeve, and a sleeve locking member, the drive shaft including a proximal end, a distal end, and a longitudinal axis extending therebetween, the distal end defining a drive mechanism engageable with the drive mechanism of the pedicle screw in torque transmitting relationship; the locking sleeve having a proximal end, a distal end, a longitudinal axis extending therebetween, and an axial through bore receiving the drive shaft, the locking sleeve being mounted to the drive shaft in relative rotating relationship, the distal end of the locking sleeve defining an engagement portion engageable with the polyaxial head in rotating locking relationship; and a sleeve locking member mounted to the drive shaft in fixed angular relationship, the sleeve locking member being translatable along the shaft between a first position in which it engages the locking sleeve in fixed angular relationship and a second position in which it is disengaged from the fixed angular relationship with the locking sleeve.
